(19) United States
(12) Patent Application Publication
(10) Pub. No.: US 2010/0016763 A1
(43) Pub. Date: Jan. 21, 2010

(54) INTRALUMINAL FLUID PROPERTY STATUS SENSING SYSTEM AND METHOD

(76) Inventor: George W. Keilman, Bothell, WA (US)

Correspondence Address: DAVIS WRIGHT TREMAINE, LLP/Seattle, 1201 Third Avenue, Suite 2200, SEATTLE, WA, 98101-3045, US

(21) Appl. No.: 12/505,378

(22) Filed: Jul. 17, 2009

Related U.S. Application Data
(60) Provisional application No. 61/081,674, filed on Jul. 17, 2008.

(51) Int. Cl. A61N 1/00; A61B 5/00

(52) U.S. Cl. 601/2; 600/309

(57) ABSTRACT

Implementations of an intraluminal fluid property status sensing system and method locate an acoustic transducer within a lumen of a biological creature to transmit ultrasound through intraluminal fluid to be reflected or otherwise affected by the fluid with subsequent reception by the same transducer. Reflection or interaction of the ultrasound with an intraluminal fluid depends upon one or more properties of the intraluminal fluid so can be used to determine status of such properties.

### CROSS-REFERENCE TO RELATED APPLICATIONS

This application claims the benefit of U.S. Provisional Application No. 61/081,674, filed Jul. 17, 2008, and incorporates by reference the U.S. Provisional Application herein in its entirety.

### BACKGROUND OF THE INVENTION

1. Field of the Invention

The present invention is generally related to fluid property sensing.

2. Description of the Related Art

Conventional ultrasound methods are known to determine fluid property status of biological fluids of a biological creature contained outside of the biological creature. For instance, ultrasonic standing waves are used to force the red blood cells to accumulate at pressure minima. By optically measuring width of resultant bands, red blood cell concentration, related to hematocrit status can be measured directly. The effects of hematocrit, shear rate, and turbulence in blood on ultrasonic Doppler spectrum, scattering of ultrasound by red blood cells, and effects of hematocrit on attenuation of ultrasonic signals transmitted through a vial containing blood have been studied.

Pulse-echo technique to measure hematocrit by measuring the attenuation of the blood contained outside of the biological creature as a function of range in front of the transducer and use of ultrasonic transducers that are in fluid contact on a surface of a liquid to sense the viscosity of the liquid have also been studied. The travel time (delay) of an acoustic signal in an external liquid sample have been used to measure temperature of the sample, while the attenuation is used to measure the viscosity of the sample.

Fluid property status of an intraluminal fluid inside a lumen of a biological creature can be assessed to a certain degree through conventional methods using ultrasonic transducers positioned outside of the biological creature. Although these conventional approaches for determining fluid property status of externally and intraluminally contained fluids using externally positioned transducers are useful, new approaches would be desirable.

### BRIEF DESCRIPTION OF THE SEVERAL VIEWS OF THE DRAWING(S)

FIG. 1 is a schematic view of a first version of an intraluminal fluid property status sensing system as positioned in a lumen.

FIG. 2 is a schematic component diagram of an intraluminal component of the first version of the system of FIG. 1.

FIG. 3 is a schematic component diagram of an external component of the first version of the system of FIG. 1.

FIG. 4 is a schematic view of a second version of an intraluminal fluid property status sensing system as positioned in a lumen.

FIG. 5 is a schematic component diagram of an intraluminal component and an external component of the second version of the system of FIG. 4.

### DETAILED DESCRIPTION OF THE INVENTION

As discussed herein, implementations of an intraluminal fluid property status sensing system and method locate an acoustic transducer within a lumen of a biological creature to transmit ultrasound through the intraluminal fluid to be reflected or otherwise affected by the fluid with subsequent reception by the same transducer. Reflection or interaction of the ultrasound with an intraluminal fluid depends upon one or more properties of the intraluminal fluid so can be used to determine status of such properties.

In different implementations, the fluid status system may be either permanently or temporarily implanted or inserted in a lumen of living beings for purposes of monitoring the hematocrit and/or other properties of the fluid. One specific application is in the measurement of fluid status within the blood (e.g., to measure hematocrit) and other physiological parameters from within a blood vessel or within the heart itself.

One implementation includes an external electronic component communicatively linked through a wireless connection via an RF magnetic field to an internal sensing component. With this implementation, the internal sensing component could be inserted or implanted within the heart or elsewhere in the vasculature (such as in a dialysis shunt). In another implementation, the external electronic component is wirelessly coupled via an acoustic link to the internal sensing component.

Other implementations include the internal sensing component on a catheter or cannula, with lead wires extending up the catheter or cannula and out of the body to external instrumentation. Another implementation includes the internal sensing component on a pacing lead, with wires running up the pacing lead, or connection tether of wires to an implantable defibrillator, pacemaker, monitoring device or combination device. The internal sensing component can either store in memory, algorithmically process and store in memory or telemeter the data directly out of the body, using RF or acoustic transmission.

The fluid status system permits continuous, real-time interrogation of fluid properties using ultrasonic transducers. In some applications, the internal sensing component with its sensors can be placed by insertion, implantation, otherwise in intravascular locations in animals or in the human body for the purpose of measuring fluid attenuation, temperature, and other physiologic parameters. Implementations can use a minimum number of components that allow the internal sensing component to be reduced to a size suitable for insertion as a component of or inside of a typical intravascular or intracardiac catheter diameter.

The fluid status system can be used without need to withdraw fluid from the patient. Fluid status can be sampled in a small region at a desired location within a subject. Samples of fluid status can be continuously taken and recorded to provide trending data. Also, the multiple parameters (such as fluid viscosity and temperature) can be sensed simultaneously with the same sensor.

Acoustic sensors can be designed to respond to changes in fluid properties. Several types of sensor arrangements can be used, depending upon the parameter being sensed and the type of acoustic wave that is being generated and detected. The fluid status system has further advantages compared with systems that have externally located sensors. Measurement accuracy issues due to the attenuation of the intervening tissue (between the transducer and the blood) are eliminated. Alignment issues between the transducer beam and the blood vessel are eliminated, because the sensor is in the blood. Fixed positioning inside the bloodstream should provide much more stable and repeatable data over a sequence of readings. The device is not hand-held, so user issues can be reduced or eliminated thereby enabling automated data acquisition. Furthermore, no acoustic coupling gel is needed.

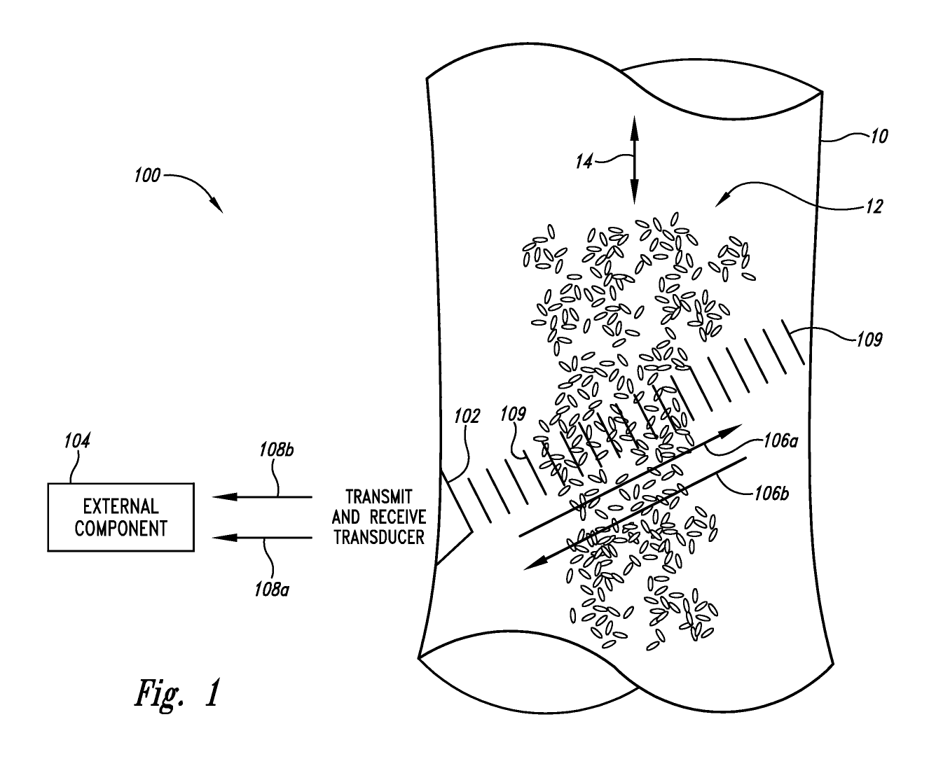

A first version 100 of the intraluminal fluid status sensing system is shown in FIG. 1 to include an internal sensing component 102 (shown implanted on an inside wall of a lumen 10) and an external component 104. In the first version 100 depicted, the sensing component 102 sends an original ultrasonic signal 106a into an intraluminal fluid (such as blood) contained in a lumen 10 (such as a blood vessel) to be reflected off of scatterers 12 (such as particles, salts, densities, colors, temperatures, fluid dynamics, chemicals, artifacts, or other properties) found in the intraluminal fluid having a flow 14 and to be subsequently received by the sensing component as the reflected ultrasonic signal 106b.

In some implementations, the original ultrasonic signal 106a can also be wirelessly transmitted to the external component 104 as an original external signal 108a (e.g. as a radio or acoustic signal) to be used for timing information by the external component 104. Outputting the original ultrasonic signal 106a to the external component 104 as the original external signal 108a is useful in situations where the sensing component 102 is “free running”, i.e., generating pulses at some rate and at some instances in time that are not controlled by the external component 104. The external component 104 can receive and “lock onto” the original external signal 108a so that the external component 104 samples the echo signals after a delay, at the appropriate points in time. In a situation where the external component 104 is wired to the sensing component 102 (such as a catheter) the external component could control the timing of the pulses so that the original external signal is unnecessary.

The sensing component 102 sends on the received reflected ultrasonic signal 106b as a reflected external signal 108b. For hematocrit measurements in particular, orientation of the original ultrasonic signal 106a with respect to direction of the fluid flow 14 can be varied to a relatively large degree.

The sensing component 102 is electrically excited to send the original ultrasonic signal 106a into the fluid as a longitudinal acoustic wave. Liquids and gases generally support longitudinal acoustic waves. Solids support longitudinal and transverse (shear) wave types, and solids with interfaces supporting additional “surface” wave types. In solids with two surfaces (such as thin plates), additional wave types can be supported. Additionally, solids immersed in fluids can support evanescent wave fields that propagate along the boundary, with the wave's travel velocity and/or attenuation being altered by the fluid characteristics.

The sensing component 102 then receives the reflected ultrasonic signal 106b as echoes from the flowing fluid 14 to convert back into an electrical signal. The time required for the original ultrasonic signal 106a to travel from the sensing component 102 to an ensemble of the scatterers 12 as scatterers (i.e., red blood cells and other blood constituents) and back as the reflected ultrasonic signal 106b to the sensing component is used to “range gate” the original ultrasonic signal and the resultant reflected ultrasonic signal.

A series of range gates 109 (also known as range cells) are depicted for attenuation measurements involved with determination of hematocrit and other fluid status. The external component 104 processes signals received from the sensing component 102 in a delayed fashion as is known in the art provide the range gating. Delays of fixed intervals are built into signal processing by the external component 104 so that various ranges of the reflected ultrasonic signal 106b distanced from one another are accounted for in fluid status determination by the external component.

Fluid status measurement, such as involving attenuation measurement, results from the measure of two (or more) echo amplitudes of the reflected ultrasonic signal 106b at different distance ranges. If attenuation of the original ultrasonic signal 106a and the reflected ultrasonic signal 106b were substantially absent, then amplitude of that portion of the reflected ultrasonic signal 106b received by the transducer 118 would change as a function of range distance of reflection of the original acoustic signal 106a occurring from the transducer, due to beam-spreading (diffraction) effects as a function of the range distance. Factors involved include aperture size, shape, and ultrasonic frequency. This effect related solely to no attenuation by the fluid 14 would be characterized for a given design, and used by the external component 104 to compensate raw measurements.

Attenuation is typically measured in dB/cm (or dB/mm) at a given frequency. Sometimes it is specified as the attenuation slope (e.g., dB/cm-MHz). If the echo amplitude is measured at two successive ranges, as A1 and A2, spaced apart by a distance Z in cm, then the “raw” attenuation is:

20*log(A2/A1)/Z in units of dB/cm.

This attenuation value (which is negative, as A2 is always smaller than A1), would then be adjusted by adding the diffraction compensation value, in order to arrive at the attenuation in the fluid itself.

The attenuation value, in dB/cm, increases with ultrasonic signal frequency. This increase is nearly linear over a narrow frequency range, and thus the attenuation slope (dB/cm-MHz) is sometimes used. This may be useful in hematocrit measurements, and if needed, it could be accomplished by stepping the oscillator frequency through several points within the passband of the transducers.

Attenuation is a combination of absorption and scattering. In blood and tissue—generally ˜90% absorption and 10% scattering. If the scattering were high, then one would not be able to make useful ultrasonic images.

Averaging would be used to improve the signal-to-noise ratio of the amplitude measurement for each range. On successive pulses, the echoes from the first range would be averaged together, and the echoes from the second range would be averaged together. The values from different ranges would not be averaged with each other.

The range gates 109 also allow for measurement of Doppler signals at multiple locations across the vessel, to obtain a flow profile, if desired. For Doppler measurements, the original ultrasonic signal 106a are aligned with direction of the fluid flow 14 improves measurement sensitivity and accuracy.

As is known, signal attenuation per unit length is used to compute fluid attenuation, which can be used to compute fluid properties such as viscosity. Viscosity can be directly related to other fluid properties, such as the concentration of cells within the fluid. If the fluid is blood, the concentration of red blood cells (termed hematocrit) can thus be measured. If the fluid is urine (instead of blood), the concentration of cells or electrolytes can be determined from the attenuation.

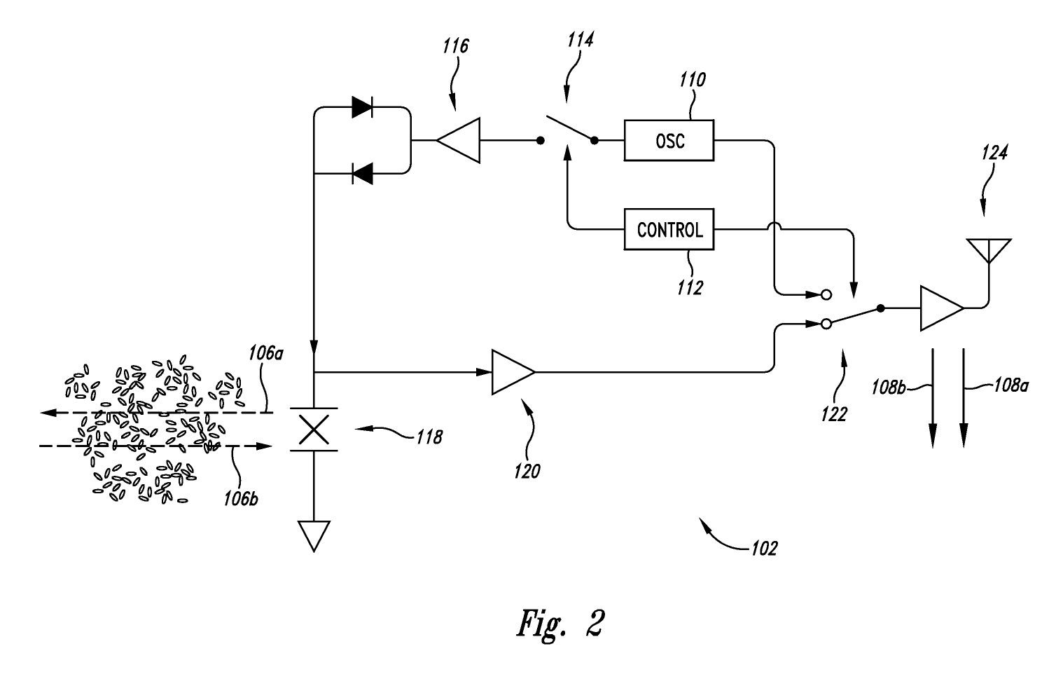

An exemplary version of the sensing component 102 is shown in FIG. 2 as including an oscillator 110, a control 112, a switch 114, an amplifier 116, a transducer 118, an amplifier 120, a switch 122 and a transmitter 124. The oscillator 110 generates an electrical signal that is sent on to the amplifier 116 when the control 112 closes the switch 114. The transducer 118 converts the electrical signal from the oscillator 110 to the original acoustic signal 116a, transmits the original acoustic signal into the intraluminal fluid 14, and subsequently receives the reflected ultrasonic signal 106b.

For 10 MHz operation, a transmit burst for the electrical signal from the oscillator 110 and subsequent original acoustic signal 106a could be 10 or 20 cycles long, so the switch 114 would be closed for 1 or 2 microseconds. The transducer 118 sends the received reflected ultrasonic signal 106b to the amplifier 120 and on to the transmitter 124 to transmit as the reflected external signal 108b when the control 112 switches the switch 122 appropriately. When the control 112 appropriately switches the switch 122, the electrical signal from the oscillator 110 is also sent to the transmitter 124 to convert and transmit as the original external signal 108a to the external component 104.

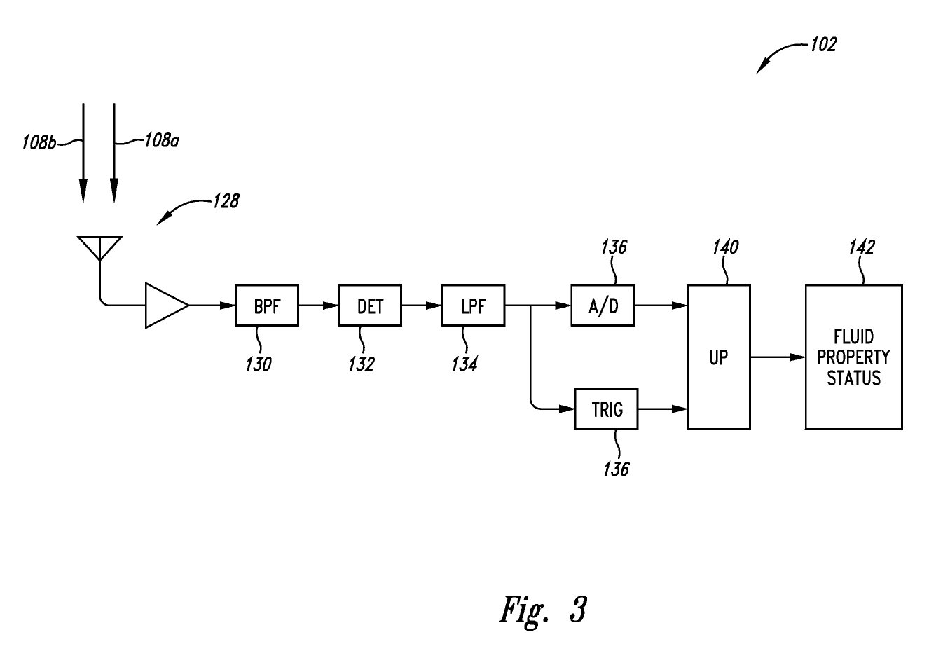

An exemplary version of the external component 104 is shown in FIG. 3 as including a receiver 128, a band-pass filter 130, a signal detector 132, and a low-pass filter 134 electrically coupled together in series. An analog-digital converter 136 and a trigger 138 are electrically coupled to the low-pass filter 104 in parallel and electrically coupled to a microprocessor 140 in parallel. The microprocessor 140 uses signal processing to output fluid property status 142 after the external component receives the original external signal 108a and the reflected external signal 108b.

The signal processing of the microprocessor 104 includes sampling the echo waveform amplitude as a function of range in front of the transducer 118. The transmit burst of the electrical signal from the oscillator 110 to generate the original acoustic signal 106a is used to produce a trigger signal, so that the analog-digital converter 136 samples with appropriate timing with respect to the transducer 118. Consequently, one or more analog-digital samples can be included within each of the range gates 109.

As echo amplitude fluctuates over time due to instantaneous variation in arrangement of scatterers within the original acoustic signal 106a, the values within each of the range gates 109 are averaged over a series of successive pulse-echo events, in order to obtain the amplitude data needed to calculate the attenuation of the signal in the fluid. Once the attenuation is determined by the microprocessor 140, the fluid property status 142 (such as hematocrit value for blood or cell/electrolyte concentration for urine) can be outputted.

In some implementations, the microprocessor 140 process echo amplitude of pulse-echo events at two or more ranges to derive attenuation values of the original acoustic signal 106a. When the intraluminal fluid 14 is blood the attenuation value can be processed by the microprocessor 140 to derive hematocrit of the blood. The sensing component 102 can be positioned within the lumen 10 to be in direct contact with the intraluminal fluid 14. The sensing component 102 can be implanted or inserted into a living being either through a natural or artificial surgically created hole in the living body.

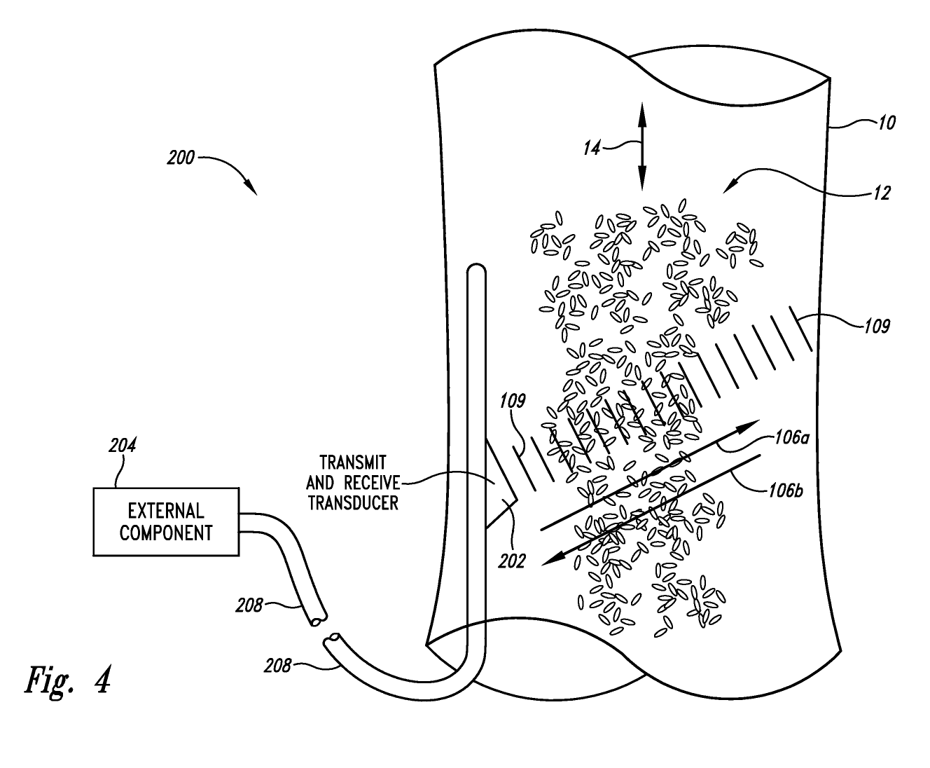

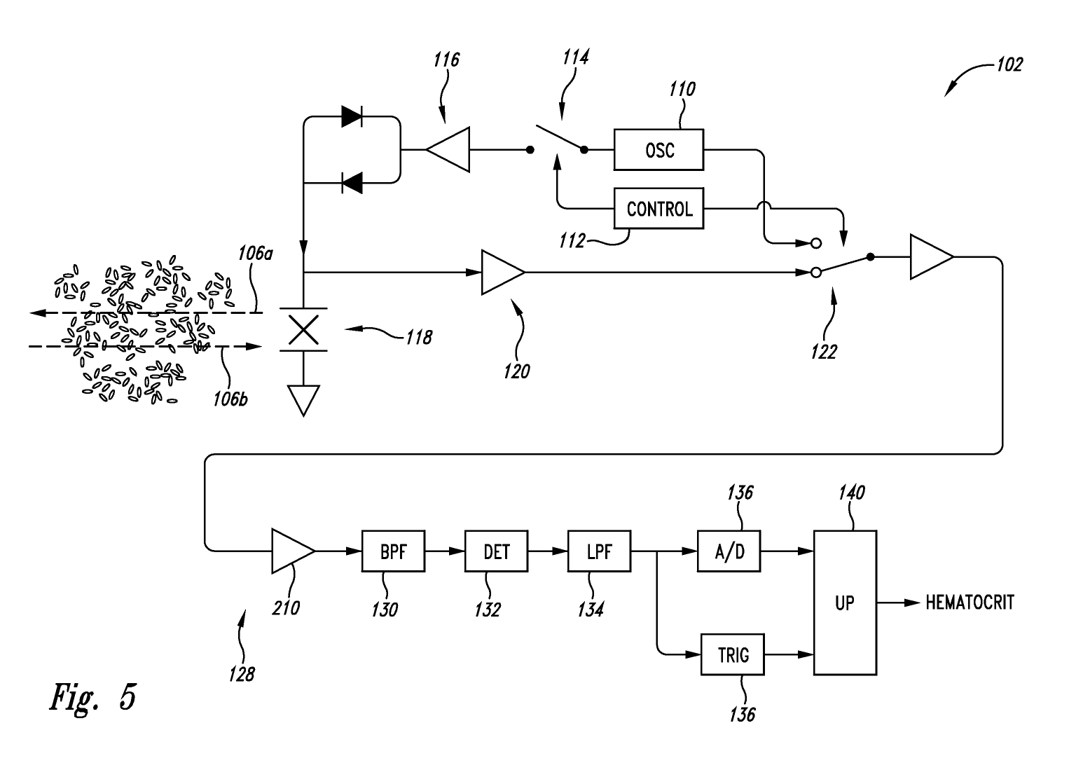

A second version 200 of the fluid status system is depicted in FIG. 4 as having an internal sensing component 202 communicatively linked to an external component 204 through a wired connection 208 such as a catheter, cannula, or pacing lead. The exemplary schematics of the sensing component 202 and the external component 204 for the second version 200 are shown in FIG. 5 to have an amplifier 210 coupled in series with the switch 122 and the band-pass filter 130.

From the foregoing it will be appreciated that, although specific embodiments of the invention have been described herein for purposes of illustration, various modifications may be made without deviating from the spirit and scope of the invention. Accordingly, the invention is not limited except as by the appended claims.

The invention claimed is:

1. A system comprising:
for positioning within a biological lumen containing a fluid, a sensing component including an oscillator and a transducer electrically coupled with the oscillator to receive electrical signaling from the oscillator, the electrical signaling to an original acoustic signal and transmit the original acoustic signal into the fluid, the transducer configured to receive and convert a first acoustic signal into a first electrical signal from the original acoustic signal being attenuated and reflected off of scatterers found in the fluid a first distance range from the transducer, the transducer configured to receive and convert a second acoustic signal into a second electrical signal from the original acoustic signal being attenuated and reflected off of scatterers found in the fluid a second distance range from the transducer, to a second electrical signal, the sensing component configured to transmit the first electrical signal as a first external signal and to transmit the second electrical signal as a second external signal; and
an external component communicatively linked to the sensing component to receive the first electrical signal and the second electrical signal, the external component including a processor to output fluid property status based upon attenuation of the original acoustic signal as indicated by the first external signal and the second external signal received by the external component.

2. The system of claim 1 wherein the sensing component is communicatively linked to the external component through a wired connection.

3. The system of claim 1 wherein the sensing component is communicatively linked to the external component through a wireless connection.

4. The system of claim 3 wherein the wireless connection is one of the following: an RF magnetic link and an acoustic link.

5. The system of claim 1 wherein the sensing component is configured to be positioned within the lumen by one of the following: insertion and implantation.

6. The system of claim 1 wherein the processor is configured to determine concentration of biological cells within the fluid as included with the fluid property status.

7. The system of claim 1 wherein the processor is configured to determine fluid property status based upon attenuation values of pulse-echo events.

8. The system of claim 7 wherein the processor is configured to determine attenuation values of the pulse-echo events through the fluid as blood to derive the fluid property status to include a hematocrit value.

9. The system of claim 7 wherein the processor is configured to average values of a series of successive pulse-echo events to obtain amplitude data to calculate attenuation of the original acoustic signal in the fluid.

10. The system of claim 7 wherein the processor is configured to process amplitudes at two or more ranges to average values of the series of successive pulse-echo events.

11. A system comprising:
for positioning within a biological lumen of a living being, the biological lumen containing blood, a sensing component including a transducer configured to transmit an original acoustic signal into the blood, the transducer configured to receive a first acoustic signal of the original acoustic signal reflected a first distance range from the transducer due to at least a hematocrit value of the blood, the transducer configured to receive a second acoustic signal of the original acoustic signal reflected a second distance range from the transducer due to at least the hematocrit value of the blood, the sensing component configured to transmit a first external signal based upon the first acoustic signal received by the transducer and to transmit a second external signal based upon the second acoustic signal received by the transducer; and
for positioning outside of the living being, an external component communicatively linked to the sensing component to receive the first external signal and the second external signal, the external component including a processor configured to output a hematocrit value based upon the first external signal and the second external signal received by the external component.

12. The system of claim 11 wherein the sensing component is communicatively linked to the external component through one of the following a wired connection and a wireless connection.

13. The system of claim 11 wherein the sensing component is configured to be positioned within the lumen by one of the following: insertion and implantation.

14. The system of claim 11 wherein the processor is configured to output the hematocrit value based upon attenuation values of pulse-echo events.

15. The system of claim 11 wherein the processor is configured to average values of a series of successive pulse-echo events to obtain amplitude data to calculate attenuation of the original acoustic signal in the fluid.

16. The system of claim 15 wherein the processor is configured to process amplitudes at two or more ranges to average values of the series of successive pulse-echo events.

17. A method comprising:
providing a sensing component having a transducer and an oscillator;
positioning the sensing component within a biological lumen of a living being, the biological lumen containing a fluid;
providing an external component having a processor;
positioning the external component outside of the living being;
switching the oscillator to momentarily couple with the transducer to transmit an original acoustic signal into the fluid within the lumen;
receiving at the transducer a first reflected acoustic signal resulting from the original acoustic signal being reflected off of scatterers contained at a first distance range from the transducer within the fluid;
sending a first external signal to the external component based on the first reflected acoustic signal received at the transducer;
receiving at the transducer a second reflected acoustic signal resulting from the original acoustic signal being reflected off of scatterers contained at a second distance range from the transducer within the fluid;
sending a second external signal to the external component based on the second reflected acoustic signal received at the transducer; and
determining with the processor of the external component an amount of attenuation of the acoustic signal with the fluid based upon the first external signal and the second external signal received by the external component.

18. The system of claim 17 wherein the sending the first external signal and the sending the second external signal are performed with a wired connection.

19. The system of claim 17 wherein the sending the first external signal and the sending the second external signal are performed through a wireless connection.

20. The system of claim 17 wherein the positioning the sensing component within in the biological lumen is performed by one of the following: insertion and implantation.

21. The system of claim 17, further including switching the oscillator for another portion of the sensing component to send an oscillator based external signal to the external component.

22. The system of claim 17 wherein the determining further includes determining concentration of biological cells within the fluid.

23. The system of claim 17 wherein the determining involves attenuation values of pulse-echo events of the original acoustic signal through the fluid as indicated by the first external signal and second external signal.

24. The system of claim 23 wherein the determining further includes using the amount of attenuation of the pulse-echo events through the fluid as blood to derive a hematocrit value of the blood.

25. The system of claim 23 wherein the determining includes averaging values of a series of successive pulse-echo events of the original acoustic signal as indicated by the first external signal and the second external signal to obtain amplitude data to calculate amount of attenuation of the original acoustic signal in the fluid.

26. The system of claim 23 wherein the determining includes processing amplitudes at two or more ranges to average values of the series of successive pulse-echo events.

27. A method comprising:
providing a sensing component;
positioning the sensing component within a biological lumen of a living being, the biological lumen containing a blood;
providing an external component;
positioning the external component outside of the living being;
transmitting an original acoustic signal into the blood from the sensing component;
receiving at the sensing component a first reflected acoustic signal resulting from the original acoustic signal being reflected off of the blood at a first distance range;
sending a first external signal to the external component from the sensing component related to the first reflected acoustic signal received at the transducer;
receiving at the sensing component a second reflected acoustic signal resulting from the original acoustic signal being reflected off of the blood at a second distance range;
sending a second external signal to the external component from the sensing component related to the second reflected acoustic signal received at the transducer; and
determining with the external component a hematocrit value for the blood based upon the first external signal and the second external signal received by the external component.

28. The system of claim 27 wherein the sending the first external signal and the sending the second external signal are performed with a wired connection.

29. The system of claim 27, further including transmitting a first external signal related to the original acoustic signal from the sensing component to the external component.

30. The system of claim 27 wherein the sending the first external signal and the second external signal is performed through a wireless connection.

31. The system of claim 27 wherein the positioning the sensing component within in the biological lumen is performed by one of the following: insertion and implantation.

32. The system of claim 27 wherein the determining involves attenuation values of pulse-echo events of the original acoustic signal through the fluid as indicated by the first external signal and second external signal.

33. The system of claim 32 wherein the determining includes averaging values of a series of successive pulse-echo events of the original acoustic signal as indicated by the first external signal and the second external signal to obtain amplitude data to calculate amount of attenuation of the original acoustic signal in the fluid.

34. The system of claim 32 wherein the determining includes processing amplitudes at two or more ranges to average values of the series of successive pulse-echo events.
